(19) United States
(12) Patent Application Publication
(10) Pub. No.: US 2003/0078579 A1
(43) Pub. Date: Apr. 24, 2003

(54) Annular repair devices and methods

(76) Inventor: Bret A. Ferree, Cincinnati, OH (US)

Correspondence Address: John G. Posa, Gifford, Krass, Groh,, Sprinkle, Anderson & Citkowski, P.C., 280 N. Old Woodward Ave., Suite 400, Birmingham, MI, 48009-5394, US

(21) Appl. No.: 10/264,157

(22) Filed: Oct. 3, 2002

Related U.S. Application Data
(63) Continuation-in-part of application No. 09/807,820, filed on Apr. 19, 2001.

(51) Int. Cl. A61F 5/04

(52) U.S. Cl. 606/53

(57) ABSTRACT

Devices are disclosed for occluding defects in an annulus fibrosis to prevent conditions such as disc herniation and recurrent disc herniation. The preferred embodiments comprise an intradiscal component having a width greater than the width of the defect and a height less than the intervertebral spacing, and an extradiscal component physically coupled to the intradiscal component, the extradiscal component having a height greater than the intervertebral spacing. The components most preferably each comprise two outwardly extending arms, of equal or unequal length, oriented transversely with respect to one another. The arms of the extradiscal component are preferably of sufficient length to overlap at least a respective portion of the adjacent vertebrae. The invention further anticipates a body disposed between the intradiscal and extradiscal components to at least partially consume the defect. The body is composed of a natural or synthetic biocompatible material, such as a resilient or compressible natural or synthetic rubber, allograft tendon, or other suitable substances. A barrier element, such as a mesh or compressible layer, or strengthening member, may further be disposed between the intradiscal component and the inner surface of the annulus fibrosis. Optionally as well, a biasing element such as a spring or tensioning cable may be inserted between the intradiscal and extradiscal components to urge them toward one another. With respect to disclosed methods, one or both of the intradiscal and extradiscal components may articulate or otherwise temporarily compact to facilitate a compressed introduction into the intradiscal space.

### REFERENCE TO RELATED APPLICATIONS

This application is a continuation-in-part of U.S. patent application Ser. No. 09/807,820, which is the U.S. national phase of PCT application Ser. No. US00/14708, filed May 30, 2000, which claims priority from U.S. patent application Ser. No. 09/322,516, filed May 28, 1999, now U.S. Pat. No. 6,245,107. The entire content of each application is incorporated herein by reference.

### FIELD OF THE INVENTION

This invention relates generally to prosthetics and, in particular, to devices for occluding intervertebral disc defects and instrumentation associated with the introduction of such devices.

### BACKGROUND OF THE INVENTION

Several hundred thousand patients undergo disc operations each year. Approximately five percent of these patients will suffer recurrent disc herniation, which results from a void or defect which remains in the outer layer (annulus fibrosis) of the disc after surgery involving partial discectomy.

In the disc of a healthy patient, the nucleus pulposus is entirely surrounded by the annulus fibrosis. In the case of the herniated disc, a portion of the nucleus pulposus has ruptured through a defect in the annulus fibrosis, often resulting in a pinched nerve. This results in pain and further complications, in many cases.

One accepted treatment involves a partial discectomy. Following such a procedure, a void remains adjacent a hole or defect in the annulus fibrosis following removal of the disc material. This hole may act as a pathway for additional material to protrude into the nerve, resulting in the recurrence of the herniation.

I have devised various solutions to this condition. Reference is made to my U.S. Pat. No. 6,245,107, the entire content of which is incorporated herein by reference, the subject matter of which resides in methods and apparatus for treating disc herniation, and recurrent disc herniation, in particular.

To correct defects of this type, a conformable device is provided which assumes a first shape associated with insertion and a second shape or expanded shape to occlude the defect. The device may take different forms according to the invention, including solidifying gels or other liquids or semi-liquids, patches sized to cover the defect, or plugs adapted to fill the defect.

The device is preferably collapsible into some form for the purposes of insertion, thereby minimizing the size of the requisite incision while avoiding delicate surrounding nerves. Such a configuration also permits the use of instrumentation to install the device, including, for example, a hollow tube and a push rod to expel the device or liquefied material out of the sheath for use in occluding the disc defect.

A device according to the invention may further include one or more anchors to assist in permanently affixing the device with respect to the defect. For example, in the embodiment of a mesh screen, the anchors may assume the form of peripheral hooks configured to engage with the vertebra on either side of the disc. The teachings further contemplates a distracting tool used to force the anchors into the vertebra. Such a tool would preferably feature a distal head portion conformal to the expanded shape of the device, enabling the surgeon to exert force on the overall structure, thereby setting the anchors.

### SUMMARY OF THE INVENTION

This invention is broadly directed to devices for occluding defects in an annulus fibrosis to prevent conditions such as disc herniation and recurrent disc herniation. Assuming the defect has a width and height, and given that the annulus fibrosis has an inner surface defining an intradiscal space between adjacent vertebrae separated by an intervertebral spacing, the preferred embodiments comprise an intradiscal component having a width greater than the width of the defect and a height less than the intervertebral spacing, and an extradiscal component physically coupled to the intradiscal component, the extradiscal component having a height greater than the intervertebral spacing.

In the most preferred embodiment the intradiscal component comprises two outwardly extending arms. The extradiscal component also preferably comprises two outwardly extending arms, these being generally transverse to the arms of the intradiscal component. The arms of the intradiscal component may be of equal or unequal length, and the arms extradiscal component are of sufficient length to overlap at least a respective portion of the adjacent vertebrae.

The intradiscal component is typically positioned adjacent the inner surface of the annulus fibrosis, with the invention further including a body disposed between the intradiscal and extradiscal components to at least partially consume the defect. The body is composed of a natural or synthetic biocompatible material, such as a resilient or compressible natural or synthetic rubber, allograft tendon, or other suitable substances. A barrier element, such as a mesh or compressible layer, or strengthening member, may further be disposed between the intradiscal component and the inner surface of the annulus fibrosis. Optionally as well, a biasing element such as a spring or tensioning cable may be inserted between the intradiscal and extradiscal components to urge them toward one another.

In terms of an inventive method, one or both of the intradiscal and extradiscal components may articulate or otherwise temporarily collapse to facilitate a compressed introduction into the intradiscal space.

### BRIEF DESCRIPTION OF THE DRAWINGS

FIG. 1 is a lateral view of the spine and a device according to a preferred embodiment of the invention;

FIG. 2 is an axial cross section of the disc with the device of FIG. 1 in position;

FIG. 3 is an enlarged lateral view of the device and a portion of the vertebrae;

FIG. 4 is a view of the anterior aspect of the spinal canal with the posterior elements of the vertebrae removed to better view the device;

FIG. 5A is a view of the device from an intradiscal perspective;

FIG. 5B is a view of the lateral aspect of the device;

FIG. 5C is a view of the device viewed the spinal canal side;

FIG. 6 is a view of the collapsed device positioned within a tube ready for insertion;

FIG. 7A is an axial view of the device in the first stage of insertion;

FIG. 7B shows the second stage of insertion;

FIG. 7C is a lateral view of the spine and device at the same stage of insertion as shown in FIG. 7B;

FIG. 7D is a lateral view of the spine with the both sets of arms fully deployed;

FIG. 8 shows an alternative embodiment of the invention in the form of a device with arms connected by a spring;

FIG. 9A is a drawing of an alternative device having the two sets of arms of the device are oriented 90 degrees to one another;.

FIG. 9B is a cross section of the device drawn in FIG. 9A;

FIG. 10 is a cross section of a tool loaded with the device of FIGS. 9A and 9B;

FIG. 11 is a sagittal view of the spine and a device according to the invention;

FIG. 12 illustrates yet a further alternative embodiment of a device having a compressible center component;

FIG. 13 is a view of a different embodiment of an intradiscal component according to the invention;

FIG. 14 shows how a longitudinal component may be recessed into the back of the vertebrae to help prevent impingement of the device on the nerves within the spinal canal;

FIG. 15A shows a spring-loaded “toggle bolt” type component used within the disc space;

FIG. 15B is a view of the device of FIG. 15A positioned within the disc space;

FIG. 16 shows how the arms of the intraspinal component can be asymmetric;

FIG. 17A depicts an optional reinforcing piece behind an intradiscal component; and

FIG. 17B shows the device of FIG. 17A placed into the disc space.

### DETAILED DESCRIPTION OF THE INVENTION

This invention resides in an annular repair device (ARD) used to prevent recurrent disc herniation as well as the extrusion of artificial disc replacements. Very broadly, the device resembles an oversized plastic connector of the type used to hold tags on clothes at department stores. The preferred embodiment includes two sets of arms oriented 90 degrees from one another. The first set of arms rests on the inside of the Annulus Fibrosis. The second set of arms rests behind the vertebrae. The device can be made of metal, plastic, rubber, and/or suitable tissue such as allograft tendon.

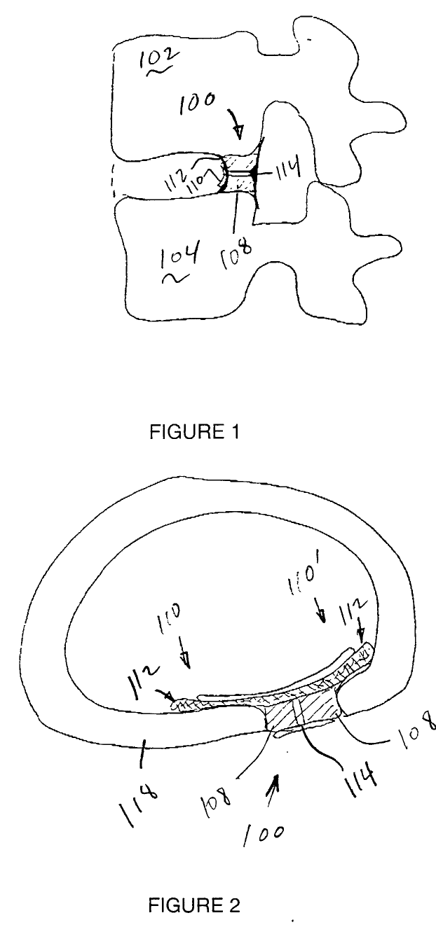

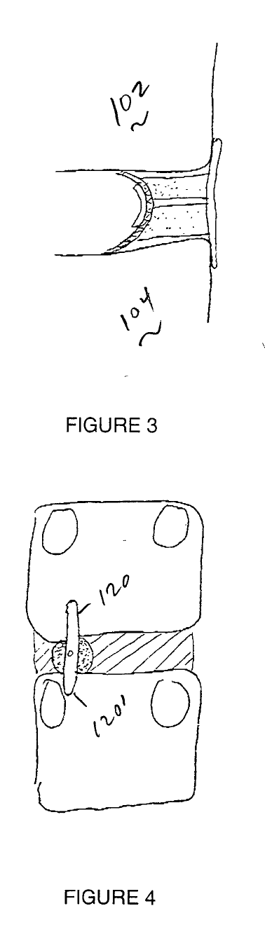

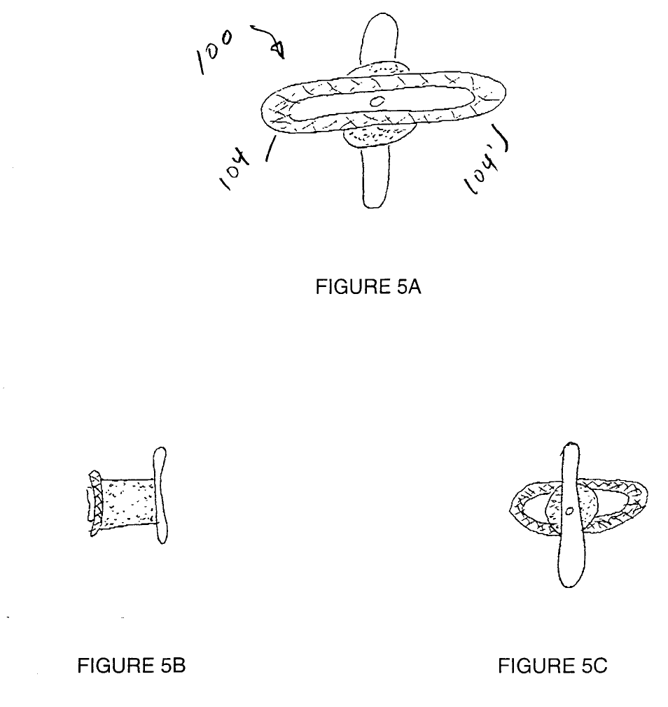

FIG. 1 is a lateral view of the spine and a device 100 according to a preferred embodiment of the invention. FIG. 2 is an axial cross section of the disc with the device 100 in position. FIG. 3 is an enlarged lateral view of the device and a portion of the vertebrae 102, 104. FIG. 4 is a view of the anterior aspect of the spinal canal with the posterior elements of the vertebrae removed to better view the device. FIG. 5A is a view of the device 100 as seen from an intradiscal perspective. FIG. 5B is a view of the lateral aspect of the device, and FIG. 5C is a view of the device from the spinal canal side.

In these figures, the diagonally hatched area 108 represents a cylinder-shaped piece of natural or synthetic material, preferably rubber or allograft tendon. Allograft tendon may aid tissue ingrowth into the device. Note that the device is not attached to either vertebra. Instead, arms 110, 110′ inside the disc are used to hold a mesh or dam 112 against the inner surface of the annulus 118, being held in position by the inner set of arms. The cylinder 108 fills the hole in the annulus. The inner arms 110, 110′ are narrow enough to allow the vertebrae 102, 104 to come closer together with spinal compression or spinal extension. The mesh or dam 112 is compressible to allow spinal motion yet prevent the extrusion of small pieces of disc material.

A connector portion 114 of the device connects the two sets of arms 110, 110′. The connector portion 114 is surrounded by the rubber or allograft cylinder 108. The connector portion 114 is coupled to a second set of arms 120, 120′, best seen in FIGS. 3, 4 and 5 which rest on the posterior aspect of the vertebral bodies. In the preferred embodiment, the geometry of the connector portion 114 bows the two sets of arms toward the connector, as perhaps best seen in FIG. 2. Thus, one set of arms pulls on the other set of arms to prevent migration of the device while the vertebrae are free to move. The arms within the spinal canal are long enough to remain positioned behind the vertebrae with distraction of the posterior portion of the vertebrae during spinal flexion.

The device is constructed for introduction through a relatively small incision.

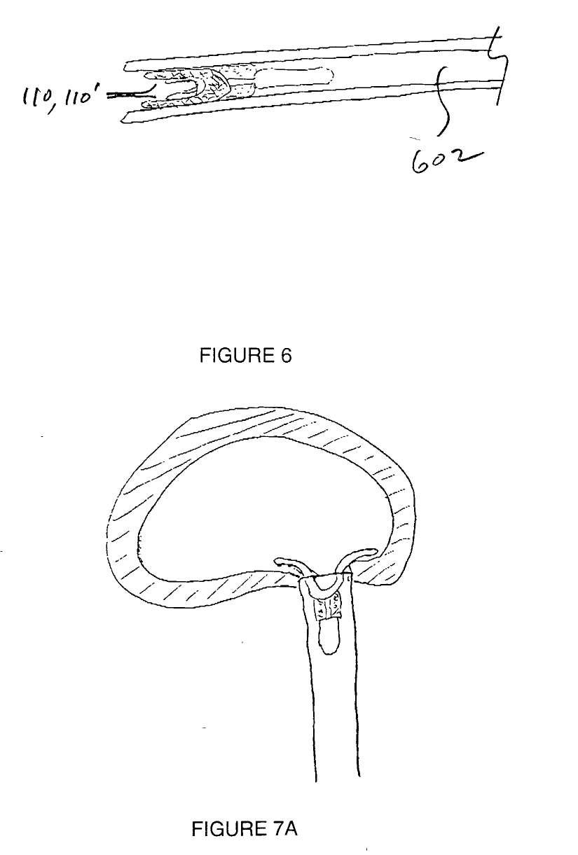

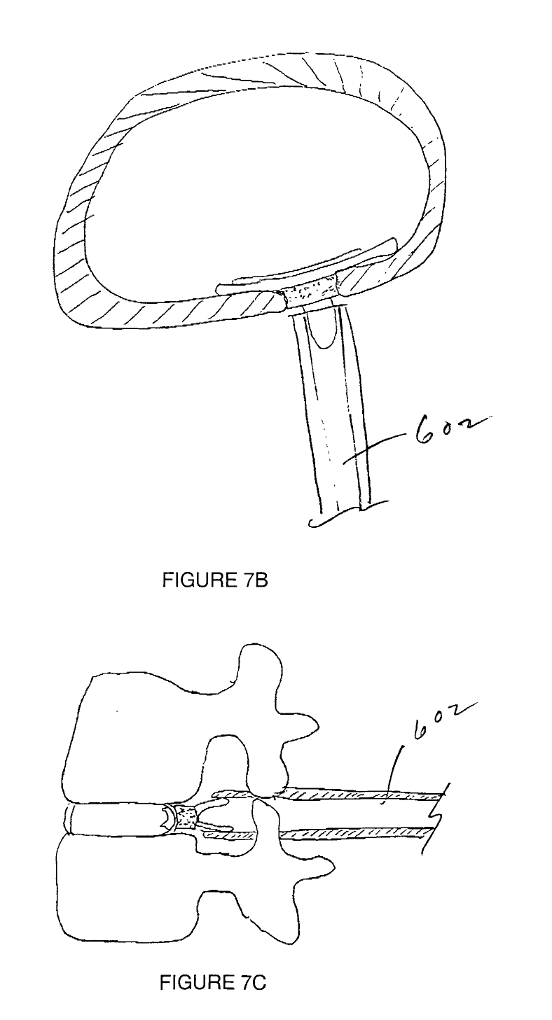

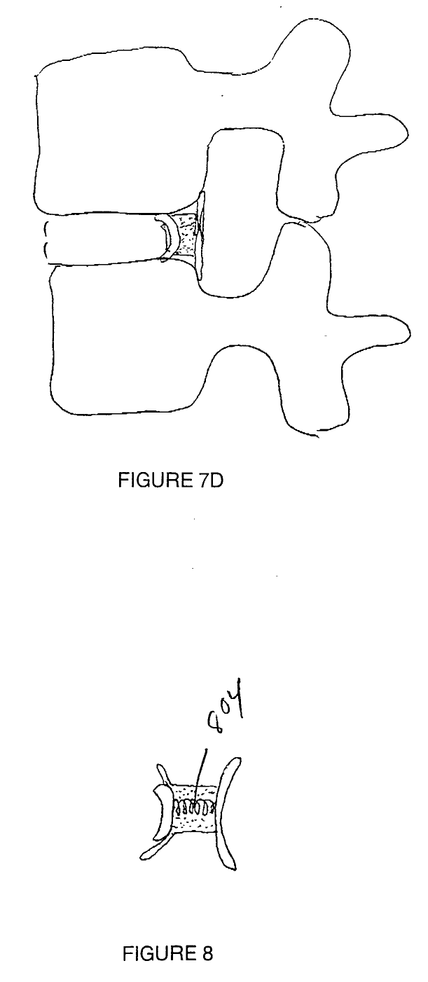

FIG. 6 shows the collapsed device positioned within a tube 602. Note how the intradiscal arms 110, 110′ may extend the opposite direction of the collapsed spinal canal arms. FIG. 7A is an axial view of the device in the first stage of insertion, wherein the intradiscal arms extend as the device is pushed from the tube. FIG. 7B shows the second stage of insertion, with he intradiscal arms are now fully deployed. The spinal canal arms 120, 102′ are still collapsed, however. FIG. 7C is a lateral view of the spine and device at the same stage of insertion as that depicted in FIG. 7B. FIG. 7D is a lateral view of the spine with the both sets of arms fully deployed.

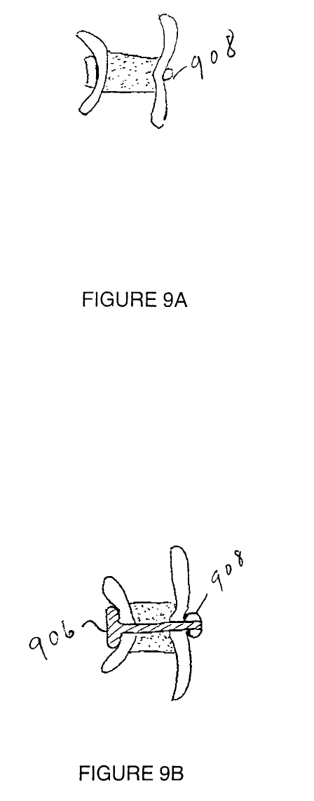

FIG. 8 shows an alternative embodiment of the device, wherein arms are interconnected with a biasing element such as a spring 804. As in the other embodiments, the embodiment of FIGS. 9A and 9B includes two sets of arms generally oriented 90 degrees to one another. FIG. 9B is a cross section of the device drawn in FIG. 9A, with the dotted area represents a component, preferably compressible, that lies within the annular hole to prevent the device from sliding up or down on the back of the vertebral bodies.

The hatched area 906 represents an elongated component or cable that extends through both sets of arms and the compressible center component. Tension is applied to the longitudinal component bowing both sets of arms. A crimp 908 on the longitudinal component holds the tension on the arms of the device.

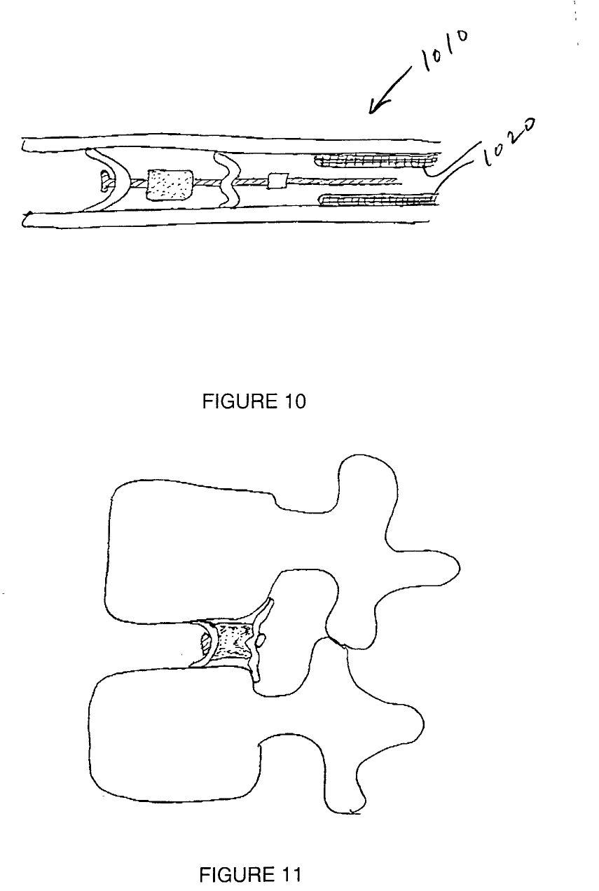

FIG. 10 is a cross section of a tool 1010 loaded with the device of FIG. 9. The area 1020 represents a portion of the tool that pushes the device out of the tool. A second portion of the tool (not drawn) pulls on the elongated component or cable. Once a sufficient amount of tension is obtained, a component of the tool deforms the crimp and cuts the longitudinal component. FIG. 11 is a sagittal view of the spine with the device of FIGS. 9 and 10 in position.

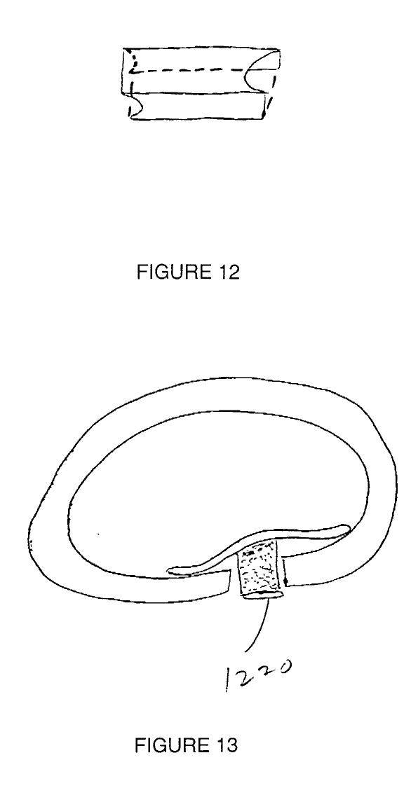

FIG. 12 is a view of an alternative embodiment wherein a central component features arms that cooperate with the sides of the arms of the longitudinal component within spinal canal. The arms of the center component would help prevent rotation of the longitudinal component within the spinal canal. Thus, the arms of the center component would keep the longitudinal component positioned behind the vertebrae.

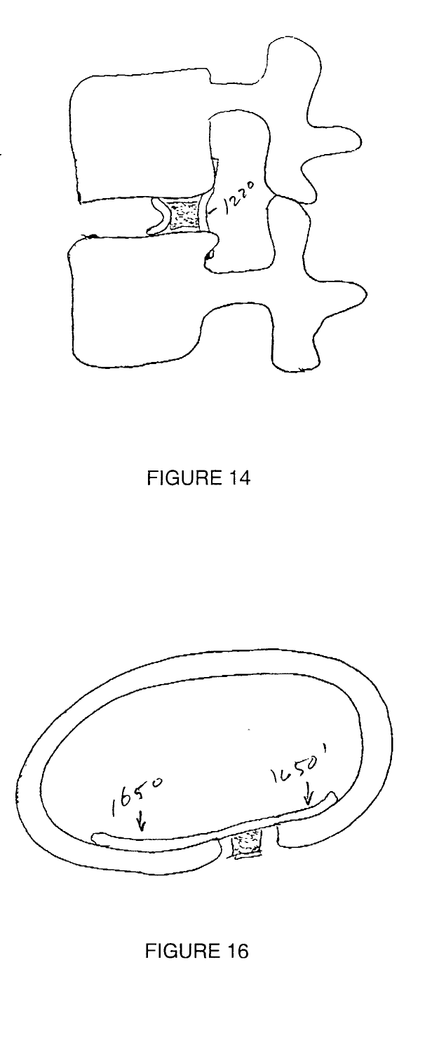

FIG. 13 is a view of an alterative embodiment of the intradiscal component of FIG. 12. Such an intradiscal component could be convex to help deflect disc material. FIG. 14 is a lateral view of the spine and the device of FIGS. 12 and 13. Note that in this and in other embodiments the longitudinal component 1220 could be recessed into the back of the vertebrae to help prevent impingement of the device on the nerves within the spinal canal.

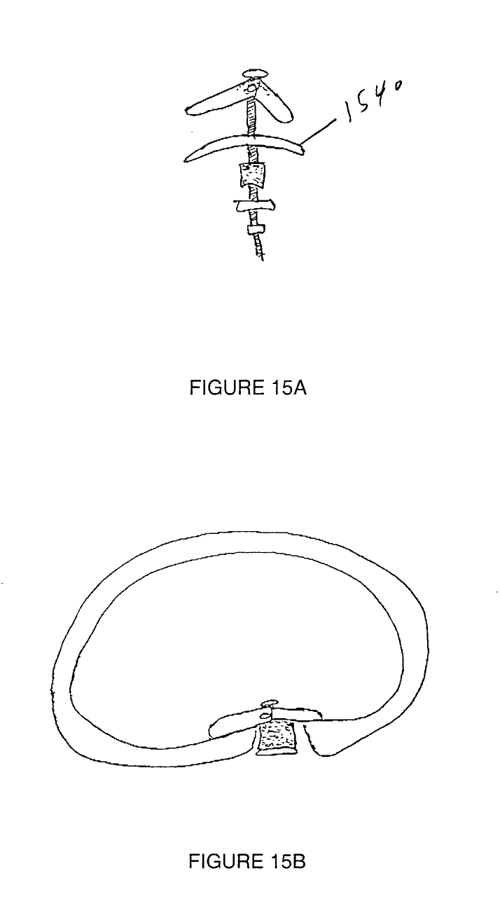

FIG. 15A shows yet a further alternative embodiment of the invention, wherein a spring-loaded element similar to a “toggle bolt” is used within the disc space to hold a mesh screen 1540 or other material over the hole in the annulus. FIG. 15B is a view of the device drawn in FIG. 15A, positioned within the disc.

FIG. 16 shows how the arms 1650, 1650′ of the intraspinal component may be asymmetric in any of the embodiments. For example, the arm that projects laterally may be shorter than the arm that projects medially. Alternatively, the lateral arm could be curved to accommodate the shape of the disc.

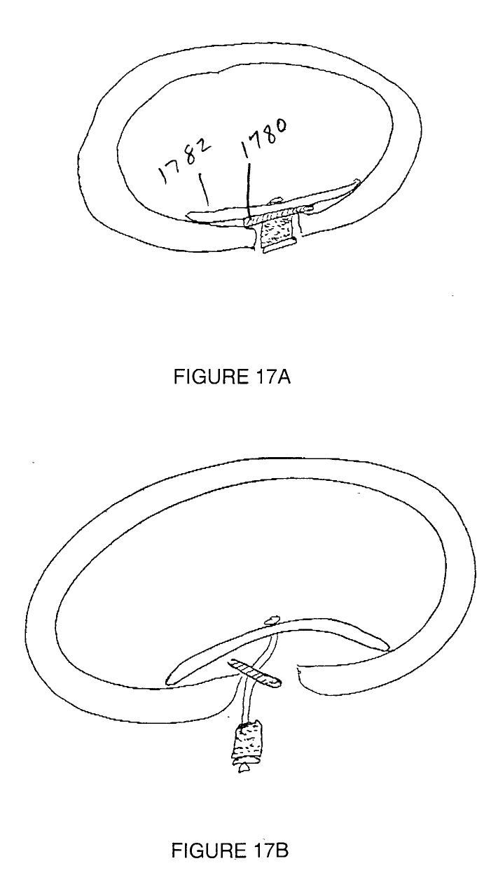

FIG. 17A is a view of an optional reinforcing piece 1780 behind the intradiscal component 1782. The reinforcing piece 1780 helps prevent bending of the intradiscal component once the device is positioned within disc space. Alternatively, the reinforcing piece could be positioned in front of the intradiscal component with arms. FIG. 17B is a view of the device drawn in FIG. 17A as the device is placed into the disc. The reinforcing piece is positioned first behind the annulus on one side of the hole in the annulus then the behind the annulus on the other side of the hole.

I claim:

1. A device for occluding a defect having a width and height in an annulus fibrosis having an inner surface defining an intradiscal space between adjacent vertebrae separated by an intervertebral spacing, the device comprising:
an intradiscal component having a width greater than the width of the defect and a height less than the intervertebral spacing; and
an extradiscal component physically coupled to the intradiscal component, the extradiscal component having a height greater than the intervertebral spacing.

2. The device of claim 1, wherein intradiscal component comprises two outwardly extending arms.

3. The device of claim 1, wherein extradiscal component comprises two outwardly extending arms.

4. The device of claim 1, wherein the intradiscal component is positioned adjacent the inner surface of the annulus fibrosis.

5. The device of claim 1, further including a body disposed between the intradiscal and extradiscal components to at least partially consume the defect.

6. The device of claim 1, wherein the body is composed of a natural or synthetic biocompatible material.

7. The device of claim 6, wherein the natural or synthetic biocompatible material is resilient or compressible.

8. The device of claim 7, wherein the resilient or compressible material is natural or synthetic rubber.

9. The device of claim 7, wherein the resilient or compressible material is tendon.

10. The device of claim 1, further including a barrier element disposed between the intradiscal component and the inner surface of the annulus fibrosis.

11. The device of claim 10, wherein the barrier element is a mesh or compressible layer.

12. The device of claim 1, further a biasing element between the intradiscal and extradiscal components operative to urge them toward one another.

13. The device of claim 12, wherein the biasing element is a spring.

14. The device of claim 12, wherein the biasing element is a tensioned cable.

15. The device of claim 1, wherein one or both of the intradiscal and extradiscal components articulate to facilitate a compressed introduction into the intradiscal space.

16. The device of claim 1, further including a strengthening element disposed between the intradiscal and extradiscal components.

17. The device of claim 1, wherein:
the intradiscal component comprises two outwardly extending arms;
the extradiscal component comprises two outwardly extending arms; and
the arms of the intradiscal and extradiscal components are substantially transverse to one another.

18. The device of claim 1, wherein:
the intradiscal component comprises two outwardly extending arms; and
the arms are of unequal length.

19. The device of claim 1, wherein:
the extradiscal component comprises two outwardly extending arms; and
the arms are of sufficient length to overlap at least a respective portion of the adjacent vertebrae.

20. A device for occluding a defect having a width and height in an annulus fibrosis having an inner surface defining an intradiscal space between adjacent vertebrae separated by an intervertebral spacing, the device comprising:
an intradiscal component with two outwardly extending arms positioned adjacent the inner surface of the annulus fibrosis, the arms defining a width greater than the width of the defect and a height less than the intervertebral spacing; and
an extradiscal component physically coupled to the intradiscal component, the extradiscal component having two outwardly extending arms defining a height greater than the intervertebral spacing.

21. The device of claim 20, further including a body disposed between the intradiscal and extradiscal components to at least partially consume the defect.

22. The device of claim 21, wherein the body is composed of a natural or synthetic biocompatible material.

23. The device of claim 22, wherein the natural or synthetic biocompatible material is resilient or compressible.

24. The device of claim 23, wherein the resilient or compressible material is natural or synthetic rubber.

25. The device of claim 23, wherein the resilient or compressible material is tendon.

26. The device of claim 20, further including a barrier element disposed between the intradiscal component and the inner surface of the annulus fibrosis.

27. The device of claim 26, wherein the barrier element is a mesh or compressible layer.

28. The device of claim 20, further a biasing element between the intradiscal and extradiscal components operative to urge them toward one another.

29. The device of claim 28, wherein the biasing element is a spring.

30. The device of claim 28, wherein the biasing element is a tensioned cable.

31. The device of claim 20, wherein one or both of the intradiscal and extradiscal components articulate to facilitate a compressed introduction into the intradiscal space.

32. The device of claim 20, further including a strengthening element disposed between the intradiscal and extradiscal components.

33. The device of claim 20, wherein the arms of the intradiscal and extradiscal components are substantially transverse to one another.

34. The device of claim 20, wherein the arms of the intradiscal component are of unequal length.

35. The device of claim 20, wherein the arms of the extradiscal component are of sufficient length to overlap at least a respective portion of the adjacent vertebrae.
